(19) United States
(12) Patent Application Publication
(10) Pub. No.: US 2011/0124094 A1
(43) Pub. Date: May 26, 2011

(54) FLUID CELL AND GENE SEQUENCING REACTION PLATFORM AND GENE SEQUENCING SYSTEM

(75) Inventor: Sitong Sheng, Guangdong (CN)

(73) Assignee: SHENZHEN CHINA GENE TECHNOLOGIES COMPANY, LTD., Guangdong (CN)

(21) Appl. No.: 13/054,249

(22) PCT Filed: Jul. 16, 2009
(86) PCT No.: PCT/CN2009/072789

§ 371 Date: Jan. 14, 2011

(30) Foreign Application Priority Data
Jul. 16, 2008 (CN) 200810132008.8

(51) Int. Cl. C12M 1/40

(52) U.S. Cl. 435/286.1; 435/289.1; 435/305.2; 435/303.1; 435/287.2

(57) ABSTRACT

In the invention, a fluid cell, used for sequencing reaction between DNA fragments and reagents, comprises: a reaction chamber, one inner side of which is fixed with multiple DNA fragments; a reagent inlet and a reagent outlet, which are separately located at each end of the other inner side of the reaction chamber and used for reagents flowing into and out from the reaction chamber. Multiple DNA fragments are fixed on the reaction chamber with the small capacity as short tag arrays in sequence and make the reaction go on at the condition of the reagents without diffusion barriers, which improves the contacts of reagents and DNA fragments so as to shorten the reaction time. At the same time, the demands to the concentration and dosage of reagents are low, which reduces the consumption of reagents. As the result, the sequencing cost cut down. Multiple DNA fragments are fixed on the fluid cell at the same time, which provides a way for parallel reaction of various fragments. The automatic high-through sequencing and fast sequencing reactions are achieved in the invention.

### FIELD OF THE INVENTION

The present invention relates to the flied of nucleotide, and more particularly to a fluid cell a gene sequencing reaction platform and a gene sequencing system.

### BACKGROUND OF THE INVENTION

Traditional Sequencing technology distinguishes the difference of DNA fragment lengths using the method of gel electrophoresis with stepwise paused polymerization process.

One of the present technologies mixes in the dideoxyribonucleoside triphosphate (ddNTP) during DNA polymerization, which lacks a hydroxyl at the 3′ position of the nucleotide, so it can not form Phosphodiester bond with the following dNTP. In the presence of ddCTP, dCTP and three other dNTP, mixing in Primers, Templates and DNA polymerase together and maintaining the temperature, the polymerization process will form a mixture of multiple fragments with different length but containing the same 5′-end primers and ddC residue on the 3′end. The various population of products in each dideoxyribonucleoside triphosphate (ddNTP) group (each has one of the four different bases labeled) are separated according to different lengths of chains, and the corresponding radioactive autoradiography or fluorescence spectra is obtained. Sequences of DNA bases can be read from the spectra. Using this technology for single gene fragment sequencing, the read length of sequences can be long. Since gene fragments must be handled individually when adding samples, the data throughput can not be improved greatly, the number is still between dozen to dozens. Moreover, it needs to separately clone each fragment, which is complicated and time-consuming.

Therefore a new technology is much needed to improve the efficiency of gene sequencing.

### BRIEF SUMMARY OF THE INVENTION

The present invention provides a fluid cell for rapid high-throughput sequencing.

In order to achieve the goal of the present invention, a fluid cell is used to carry out the reaction between DNA fragments and reagents. The present invention comprises: a reaction chamber, one inner side of which is fixed with multiple DNA fragments; a reagent inlet and a reagent outlet, which are separately located at each end of the other inner side of the reaction chamber and used for reagents flowing into and out from the reaction chamber.

Wherein, the fluid cell comprises: a drilled cover slide, a slide which is parallel and opposite to the drilled cover slide, and a gasket between the drilled cover slide and the slide; the gasket has through-holes in its central section, and is pressed closely with the drilled cover slide and the slide on its two sides separately; the through-holes, inner side of the drilled cover slide and inner side of the slide form the closed reaction chamber; as one side of the reaction chamber, the inner side of the slide is fixed with DNA fragments; as the other side of the reaction chamber, two ends of the drilled cover slide have the first and second through-hole, which are used for connecting the reaction chamber with outside separately, and form the said reagent inlet and reagent outlet.

Wherein: the through-hole of the gasket is narrow on both ends, and wide in middle, which looks like a leaf; said both ends of the through-hole separately correspond to the first through-hole and the second through-hole of the drilled cover slide, forming a reagent inlet and a reagent outlet.

Wherein: in the reaction chamber, the one side and/or the other side which fixed with DNA fragments are transparent.

Wherein: DNA fragments are attached to at least one bead which is fixed on the inner side of reaction chamber.

Wherein: DNA fragments are directly fixed on one inner side of the reaction chamber.

In order to achieve the invention's aims better, the present invention also provides a gene sequencing reaction platform for controlling sequencing reaction between the DNA fragments and reagents, which platform comprises: a fluid cell, the inner side of which is fixed with multiple DNA fragments; a temperature control unit, which is used to heat and control the temperature of sequencing reaction; a reagent control unit, which is used to control the reagents of sequencing reaction.

Wherein, the temperature control unit comprises a heating component and a temperature-measuring component: the temperature-measuring component measures temperature of sequencing reaction in real-time mode, and the measured temperature is used to control the heating component to heat the fluid cell; the heating component heats the fluid cell on the side which is fixed with DNA fragments, or on the other side.

Wherein, further comprising heating glasses which are used to heat the sequencing reaction; said heating glasses cover the outside of the fluid cell, which is heated by the heating component, and then heat and control temperature of the sequencing reaction in the reaction chamber through heat conduction of the fluid cell.

wherein the reagent control unit comprises: a reagent device which stores at least one kind of reagent; a reagent valve used for selecting one kind or several kinds of stored reagents from the reagent device; a reagent-injection component, which injects selected reagents into the fluid cell; a reagent-ejection component, used for ejecting reacted reagents from the fluid cell.

Wherein: the said fluid cell is located at level, upright or oblique.

In order to achieve the purpose of the invention better, the invention also provides a gene sequencing system for controlling the sequencing reaction between DNA fragments and reagents, and collecting and processing the data of sequencing reaction, which system comprises: a fluid cell, fixed with multiple DNA fragments and used for the gene sequencing reaction; an imaging unit, used for imaging the signal on the said sequencing DNA fragments; a data-collecting unit, used for collecting the image data; control units, used for controlling the sequencing reaction and processing image data.

Wherein the fluid cell comprises: a reaction chamber, in which multiple DNA fragments are fixed on one inner side; a reagent inlet and a reagent outlet, which are separately located at each end of the other inner side of the reaction chamber and used for reagents flowing into and out from the reaction chamber.

Wherein: the DNA fragments attached to at least one bead which is fixed on one inner side of the fluid cell; or the DNA fragments are directly fixed on the inner side of the fluid cell.

Wherein the control units comprise: a temperature control unit, used to heat and control temperature of the sequencing reaction; a reagent control unit, used to control the reagents, comprising selecting, injecting and/or ejecting; a data-processing unit used to send the control orders to the temperature unit and the reagent control unit and manage, analyze and process the image data.

Wherein the temperature control unit further comprises a heating component and a temperature-measuring component: the temperature-measuring component measures temperature of sequencing reaction in real-time mode and sends the measured temperature to the data-processing unit to analyze the measured temperature and generate temperature controlling orders; the heating component heats the fluid cell according to the temperature controlling orders from the data-processing unit.

Wherein the imaging unit comprises: a light source, which is fixed on one side of the fluid cell; an imaging component and a compound lens, which are located on the same side of the fluid cell; the imaging component takes pictures of the fluid cell through the compound lens.

Wherein: the fluid cell is located at level, upright or oblique.

Wherein the gene sequencing system further comprises: a supporting unit, used for anchoring and/or arranging the fluid cell and the imaging unit separately, in order to make the gene sequencing system stable.

Wherein the supporting unit further comprises: a position adjusting device, used for changing the distance between the fluid cell and the imaging component.

In the present invention, lots of DNA fragments fixed on small volume reaction chamber as short tags arrays in sequencing, which makes sequencing reaction processes at the condition of reagents without diffusion barrier, resulting in improving the contact between reagents and DNA fragments and shortening the time of reaction. The demands on concentration and dose of reagent are low, that is to say the consumption of reagent is less and the cost of sequencing is lower. With multiple DNA fragments fixed on a reaction chamber at the same time, it provides a platform for parallel reaction of lots of DNA fragments. This invention makes high-through sequencing automatic come into truth, integrates reaction temperature controlling, reagent controlling, imaging, data collection and procession and so on. It achieves quick sequencing reaction. Moreover, the much more through of sequence is read and efficiency is high.

### BRIEF DESCRIPTION OF THE DRAWINGS

FIG. 1 is the structure diagram of the fluid cell in the first embodiment of the present invention.

FIG. 2 is the detail structure diagram of the fluid cell in the second and the seventh embodiments of the present invention.

FIG. 3 is the exploded structure diagram of the reaction chamber in the second embodiment of the present invention.

FIG. 4 is the structure diagram of the components of gene sequencing reaction platform in the third embodiment of the present invention.

FIG. 5 is the detail structure diagram of the gene sequencing reaction platform in the third embodiment of the present invention.

FIG. 6 is another detail structure diagram of gene sequencing reaction platform in the fourth embodiment of the present invention.

FIG. 7 is the structure diagram of the components of gene sequencing system in the fifth embodiment of the present invention.

FIG. 8 is the structure diagram of the control units of the fifth embodiment in FIG. 7.

FIG. 9 is the detail components structure diagram of the gene sequencing system in the sixth embodiment.

FIG. 10 is the part components structure diagram of the gene sequencing system in the seventh and the eighth embodiments.

According to the embodiments and the figures to further explain the achievements of goal, features and advantages of the present invention.

### DETAILED DESCRIPTION OF THE INVENTION

By way of fixing DNA fragments in fluid cell to form short tags array in sequencing in the present invention, reagents directly flow through DNA fragments, therefore avoiding diffusion barrier and achieving the sequencing reaction of reagent and DNA fragments.

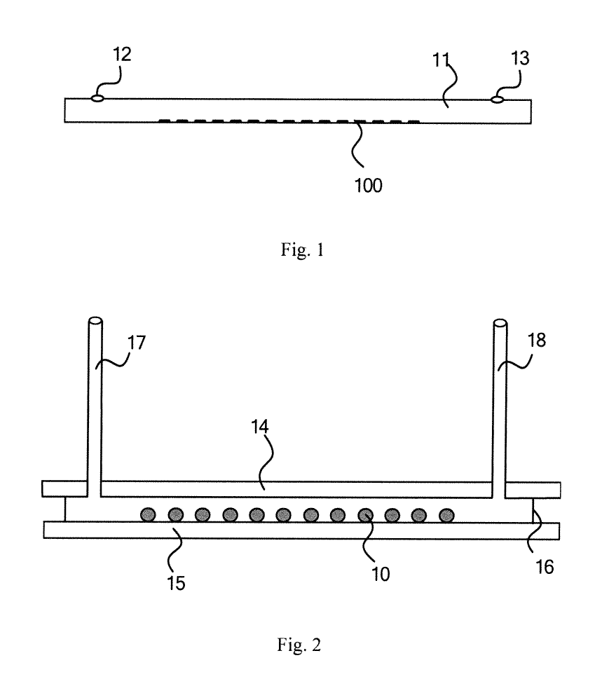

The first embodiment is proved in the invention. According to the design scheme of fluid cell in FIG. 1, the fluid cell 1 comprises the reaction chamber 11, the reagent inlet 12 and the reagent outlet 13. The DNA fragments 100 are fixed on the one side of reaction chamber 11 inner side. The reagent inlet 12 and the reagent outlet 13 are separately set at the other side of the two ends of reaction chamber 11. The reagent inlet 12 is used for reagents flowing into reaction chamber 11. The reagent outlet 13 is used for reagents flowing out from reaction chamber 11.

The one side and/or the other side of reaction chamber 11 for fixing DNA fragments 100 are transparent.

The fluid cell 1 is used for gene Sequencing and the DNA fragments 100 are fixed in reaction chamber 11 for reagents going through the reagent inlet 12 to reaction chamber 11 and reacting sequencing reaction with the DNA fragments 100. The DNA fragments 100 which have already reacted are observed and/or imaged from the one side of fixed DNA fragments 100 in reaction chamber 11. The reagents after the sequencing reaction flow out from reaction chamber 11 through reagent outlet 13.

The DNA fragments 100 can be separately fixed on one inner side of reaction chamber 11 in the embodiment and also attached to at least one bead, which is fixed on the inner side of reaction chamber 11.

After DNA fragments 100 is fixed on one inner side of reaction chamber 11, a cover made by soft material overlaps the surface. There is at least one array made by series of small holes on the cover, for the purpose of forming independent reaction chambers, where the DNA molecules amplify separately and form addressable DNA fragments 100 array on the surface. This surface fixed with DNA fragments 100 is the right one inner side of reaction chamber 11. The DNA fragments 100 are attached to at least one bead which is fixed on the inner side of reaction chamber 11, which fixes the DNA fragments 100 on the discrete solid surfaces by the method marking up biotin at the end of DNA chain. For example, the DNA fragments 100 are fixed to isolated sites or individual beads on the surface. The isolated sites or individual beads are fixed on the inner sides of reaction chamber 11 in order to form DNA array of at least one sites.

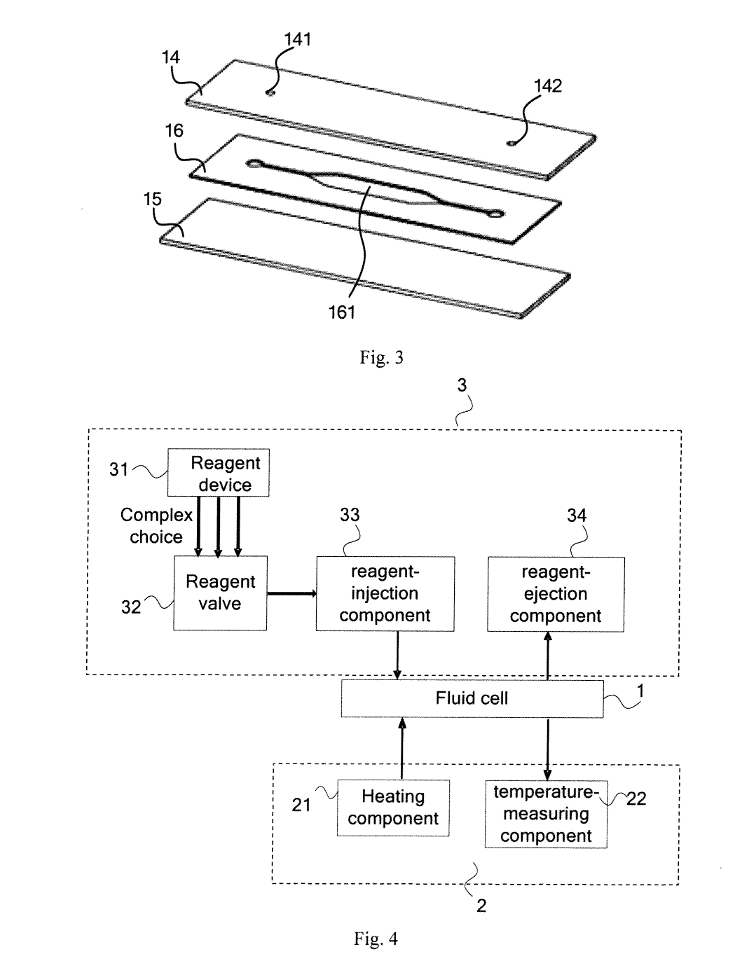

Based on the first embodiment, the invention proves the second embodiment and takes DNA fragments fixed on beads for example. FIG. 2 shows the detail structure diagram of fluid cell 1. The fluid cell 1 comprises: a drilled cover slide 14, the slide 15, the two of which is parallel and opposite, and the gasket 16 lies between the drilled cover slide 14 and the slide 15. According to the FIG. 3 about exploded structure diagram of the reaction chamber 11, there is a through-hole 161 in the center of the gasket 16. The both sides of gasket 16 closely paste the drilled cover slide 14 and the slide 15. The through-hole 161, the inner side of the drilled cover slide 14 and the slide 15 form the closed reaction chamber 11. As one inner side of reaction chamber 11, the inner side of the slide 15 has lots of beads 10 on which fixed with the DNA fragments 100 (Not shown in figure). As the other inner side of reaction chamber 11, the two ends of the drilled cover slide 14 has the first through-hole 141 and the second through-hole 142, which make the reaction chamber 11 conduct with outside, forming reagent inlet 12 and reagent outlet 13.

In this embodiment, according to pre-set, beads 10, at least one site fixed on the inner side of the slide 15, form the enriched beads arrays or plane lattices. It should be noticed that the beads 10 can be make by many kinds of materials, such as glass, plastic, metal and so on.

Drilled cover slide 14 chooses smooth slides or quartz plates, using the technology of laser or mechanical drilling to form the first through-hole 141 and the second through-hole 142. The slide 15 uses the smooth slides. The drilled cover slide 14 and/or the slide 15 are transparent. The gasket 16, the tow sides of which attach with the drilled cover slide 14 and the slide 15, using the self-adhesive gasket and softness macromolecule material is preferred, such as Silicone, rubber, PDMA, PS and so on. The through-hole 161 of gasket 16 shows the leaf shape with the two narrow ends and the wide middle. The two narrow ends of through-hole 161 is corresponding with the first through hole 141 and the second through-hole 142 of the drilled cover slide 14 and form the reagent inlet 12 and reagent outlet 13. The gasket 16 has a large area connecting with the drilled cover slide 14 and the slide 15, forming a hermetic space at the condition of non-pressure.

The fluid cell 1 can get different capacity and size by changing the thickness of the gasket 16 and the area of the through-hole 161 in this embodiment. The thickness of the gasket 16 can be chose according to demands and the optional range is from 0.125 mm to 0.5 mm. The area of the through-hole 161 can be also chose according to demands and 240 mm² is a preferential choice. The optional capacity of formed reaction chamber 11 is 60 ml.

The optional area of thought-hole 161 is 240 mm² in the embodiment, which is enough to accommodate more than 5 million beads 10 with 5 um diameter, or 16 million plastic beads with 3 um diameter, or 1.2 billion beads with 1 um diameter at the fill rate of 50%. The thickness of 250 um to the gasket 16 and the area of 240 mm² to the through-hole 161 is the optional value, which forms the optional accommodation of 60 ml to reaction chamber 11. Expensive reagents (such as Ligase and Fluorescent oligonucleotides, Polymerase and Fluorescent nucleotide) can be maximized to use in the embodiment.

The capacity of reaction chamber 11 can be chose in order to meet practical requirements in the embodiment. To change the capacity of reaction chamber 11 is just need to change the size of the gasket 16. Usually, pre-experiments of sequencing uses only small amount of reagent to make sure the feasibility of experiments, so it is favorable to do pre-experiments of sequencing. Because the capacity of the reaction chamber 11 is small, without diffusion barrier and the speed of sequencing reaction is fast, the reagent consumption of sequencing reaction is small and less than 10% of the present technology solutions. As a result, the cost of sequencing is further reduced.

In order to be easier to inject the reagents, in the embodiment the inlet pipe 17 and the outlet pipe 18, separately connecting with the reagent inlet 12 and the reagent outlet 13, is installed, which makes the reagents flow into the reaction chamber 11 and the reagents flow out from the reaction chamber 11 successfully.

Using the fluid cell 1 to gene sequencing has the similar principle of working with the previous embodiment. The detail is that many beads 10 are fixed on the inner side of the slide 15. The reagent goes along the inlet pipe 17, and goes through the reagent outlet 13, and flows into the reaction chamber 11, then comes into contact with the DNA fragments 100 on beads 10, thus starts the sequencing reaction. DNA fragments 100 of occurring sequencing reaction are observed and/or image through the slide 15 and/or bleed cover slide 14. The reagent, after sequencing reaction, goes through the reagent outlet 13 and passes the outlet pipe 18 and then flows out from reaction chamber 11.

The third embodiment is proved in the invention. FIG. 4 shows a gene sequencing reaction platform which comprises the fluid cell 1, the temperature control unit 2 using to heat and control the temperature for sequencing reaction and a reagent control unit 3 to control the reagent of sequencing reaction.

The temperature control unit 2 comprises: the heating component 21 and the temperature-measuring component 22. The temperature-measuring component 22 measures the temperature of sequencing reaction in real-time mode. The measured temperature is used for controlling the heating component 21 to heat the fluid cell 1.

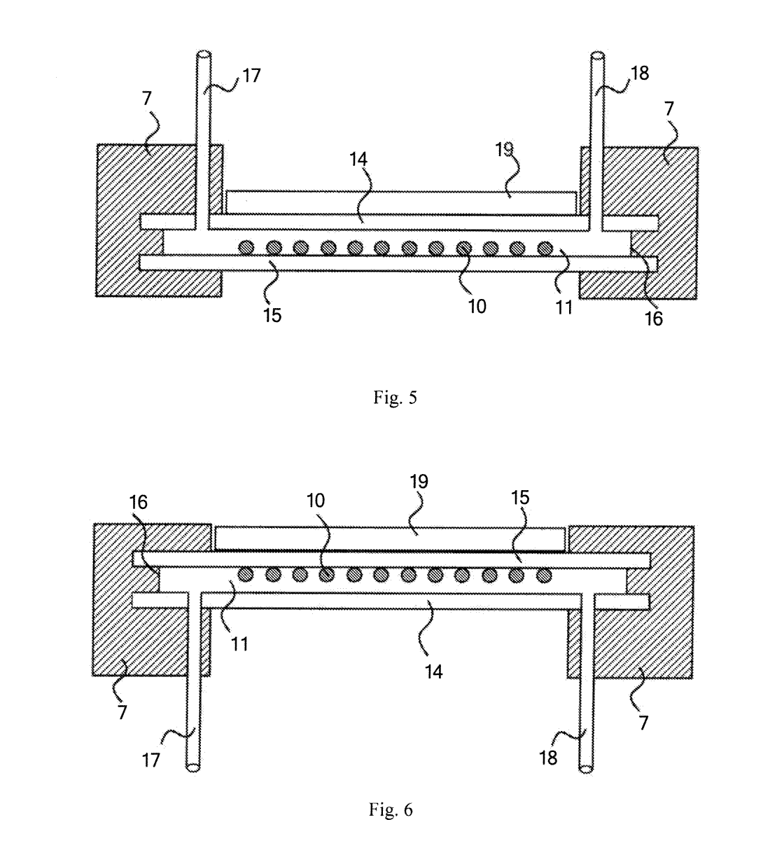

In order to control the sequencing temperature, based on the previous embodiments using a heating element 19 to heat sequencing reaction is proved in the embodiment. FIG. 5 shows the detail structure diagram of gene sequencing reaction platform. The heating element 19 which covers the outside of fluid cell 1 is heated by heating component 21, which makes the sequencing reaction in reaction chamber 11 be heated and controls the reaction temperature through the heat conduction of fluid cell 1. The option to the heating element 19 is tin indium oxide (ITO) coated glass.

The reagent control unit 3 comprises: a reagent device 31, a reagent valve 32, a reagent-injection component 33 and a reagent-ejection component 34. The reagent device 31 stores at least one kind of reagent. The reagent valve 32 selects one kind of reagent or more from the various reagents. The component 33 injects the selected reagent to the reaction chamber 11 of fluid cell 1 through inlet pipe 17. The reagent-ejection component 34 ejects the reagent reacted sequencing reaction from the fluid cell 1 going through the outlet pipe 18.

The reagent injection component 33 and/or the reagent-ejection component 34 choose the syringe liquid pumps in the embodiment.

Preferably, in the embodiment the fluid cell 1 comprises: the drilled cover slide 14, the slide 15, the two of which is parallel and opposite, and the gasket 16 located between the drilled cover slide 14 and the slide 15. In FIG. 5, the center of the gasket 16 has the through-hole 161. The two sides of gasket 16 tightly glue the drilled cover slide 14 and the slide 15. The through-hole 161, the inner side of the drilled cover slide 14 and the inner side of the slide 15 form the closed reaction chamber 11. One inner side of the slide 15 fixed on multiple DNA fragments 100 (Not shown in figure) or the beads attached multiple DNA fragments (following called beads 10 for short) as to be one inner side of reaction chamber 11. In the embodiment, the heating element 19 attaches to the outside of the drilled cover slide 14 of fluid cell 1. The heating element 19 which is heated by the heating component 21 heats for the sequencing reaction of reaction chamber 11 by the heat-conduction of the drilled cover slide 14 and controls the reaction temperature. This is to say that the heating element 19 heats the side of fluid cell 1 which does not fix DNA fragments 100 or beads 10.

The heating element 19 is made by the tin indium oxide (ITO) coated glass. The drilled cover slide 14 is made by slide or quartz plate to achieve the good heat conduction.

Using the gene sequencing reaction platform for gene sequencing, the detail is that multiple DNA fragments 100 or beads 10 are fixed in the fluid cell 1. The reagent valve 32 selects the reagent from the reagent device 31 and the reagent-injection component 33 injects reagents to fluid cell 1 through the inlet pipe 17. According to temperature measured by the temperature-measuring component 22, the heating component 21 heats the heating element 19 to control the temperature of sequencing reaction. The reagent in reaction chamber 11 reacts sequencing reaction with DNA fragments 100 and the DNA fragments of reacted sequencing reaction is observed and/or imaged from fluid cell 1. The reagent-ejection component 34 ejects the reagent reacted the sequencing reaction to the fluid cell 1 by the outlet pipe 18.

The heating element 19 and the drilled cover slide 14 have some degree of stability and good heat conduction in the embodiment, which can be used several times in sequencing reaction and make the cost of sequencing reaction further reduced. The heating element 19 and the drilled cover slide 14 have the good transmission of light so it is beneficial to observer and/or image the DNA fragments reacted. Using the embodiment to react the sequencing reaction, an ordinary power of the mercury short-arc lamp can be used as the light source and an ordinary imaging device can be use to observe and/or image, which makes the sequencing cost greatly reduced.

Based on the above embodiments, the fourth embodiment is provided in the invention. The reagent and the temperature to control sequencing reaction are posed so as to collect and process the image data.

A gene sequencing reaction platform comprises: the fluid cell 1, the temperature control unit 2 to heat for sequencing reaction and control temperature, and the reagent control unit 3 to select, inject and/or eject the reagent of sequencing reaction according to the described control units.

In the FIG. 6, compared with the third embodiment, the heating element 19, in this embodiment, tightly glues on the outside of slide 15 of the fluid cell 1. The heating element 19 which is heated by heating component 21 heats for the sequencing reaction reacting in the reaction chamber 11 and controls the reaction temperature, which is achieved by the heat conduction of slide 15. This is to say that the heating element 19 heats the side of fluid cell 1 fixed with multiple DNA fragments 100 (not shown in the figure) or beads 10. The option to the heating element (heating element) 19 is tin indium oxide (ITO) coated glass. The clear glass or quartz plate is used for slide 15 so as to achieve good heat conduction.

The heating element 19 and slide 15 have some degree of stability and good heat conduction in the embodiment, which can be used multiple times in sequencing reaction and make the cost of sequencing reaction further reduced. The heating element 19 and the slide 15 have good transmission of light so it is beneficial to observer and/or image the DNA fragments reacted. Using the embodiment to carry out the sequencing reaction, an ordinary power of the mercury short-arc lamp can be used as the light source and an general imaging device can be used to observe and/or image, which makes the sequencing cost greatly reduced.

The embodiment further comprises a pedestal 7 for fixing the fluid cell 1 and keeping its stability so that the sequencing reaction goes smoothly.

The fluid cell 1 in this embodiment and the above embodiment can be located at level, upright or oblique. For example, the fluid cell 1 in the second embodiment has the drilled cover slide 14 and the slide 15, the two of which is parallel and opposite, which can be located at level, upright or certain angle so as to make the reagent inlet 12 and reagent outlet 13 at the same level. In order to improve the result of imaging, the fluid cell 1 is located at upright in this embodiment. Reagents inject from one end of reaction chamber 11 and eject from the other end. The described one end can be higher than the other and also can be lower.

Preferably, the reagents inject from the lower end of reaction chamber 11 and eject from the higher end, which fully fills the reaction chamber 11 and contacts with DNA fragments 100 to make sure the DNA fragments 100 and the reagents fully react in the sequencing reaction. As a result, the quality of image data is much higher.

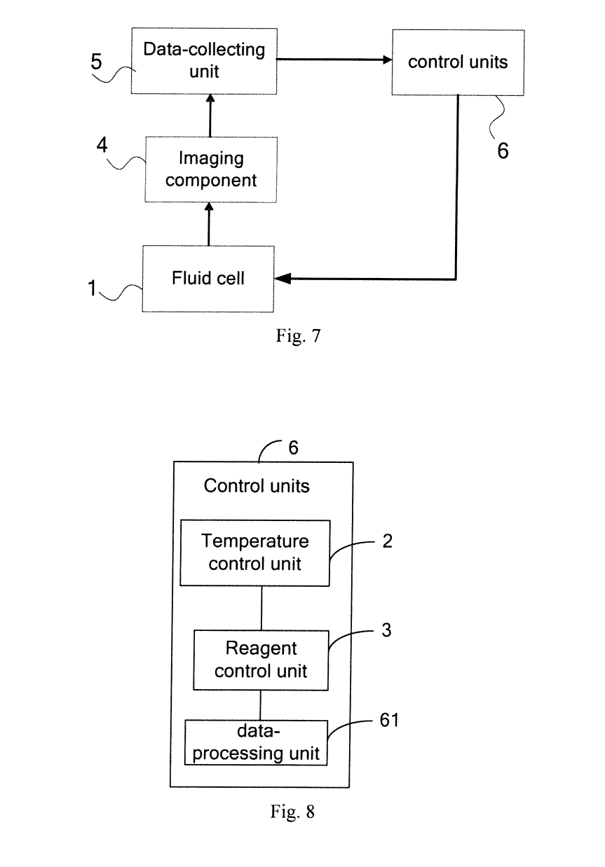

The fifth embodiment is posed in the invention. The FIG. 7 shows the gene sequencing system which comprises: the fluid cell 1 fixed with multiple DNA fragments and used to react gene sequencing reaction, the imaging unit 4 used to observe and/or image the sequencing reaction, the data-collecting unit 5 used to collect the image data and the control units 6 used to control the temperature and/or reagents of sequencing reaction and process the collected data. The imaging unit 4 collects the image data from the sequencing reaction in the fluid cell 1. The data-collecting unit 5 sends the gained image data to the control units 6. And then, the control units 6 manage, analyze and process the image data.

FIG. 8 shows the structure of control units 6 which comprise: a temperature control unit 2, a reagent control unit 3 and data-processing unit 61. The temperature control unit 2 heats for the sequencing reaction and controls the temperature. The reagent control unit 3 controls the reagent of sequencing reaction, comprises selecting, injecting and/or ejecting. The data-processing unit 61 manages, analyzes and processes the image data, and then sends the control orders to the temperature control unit 2 and the reagent control unit 3.

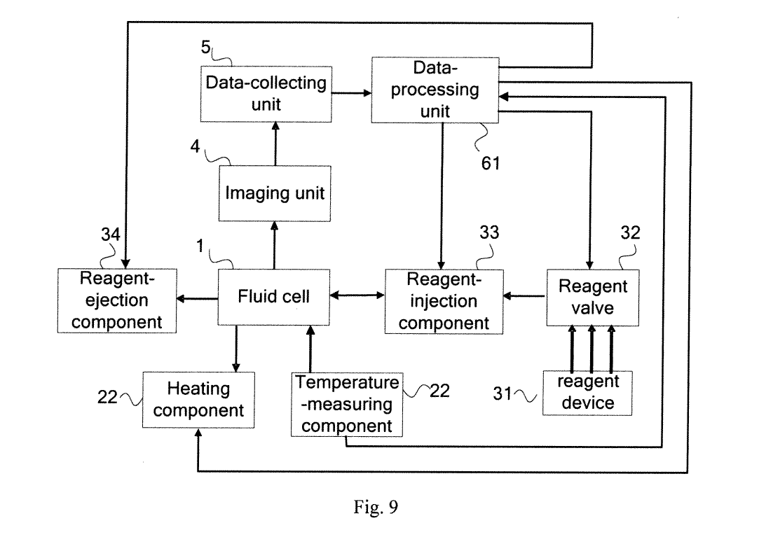

Based on the above embodiments, the sixth embodiment is posed in the invention. The FIG. 9 shows the gene sequencing system. In this embodiment, the temperature control unit 2 comprises: the heating component 21 and the temperature-measuring component 22. The temperature-measuring component 22 measures the temperature of sequencing reaction and then sends it to the data-processing unit 61. The data-processing unit 61 analyzes measured temperature, and generates temperature controlling orders, and then sends out the temperature controlling orders. The orders are sent to the heating component 21 which heats for the fluid cell 1 and uses the tin indium oxide (ITO) coated glass to heat.

In the embodiment, the reagent control unit 3 comprises: a reagent device 31, a reagent valve 32, a reagent-injection component 33 and a reagent-ejection component 34. The reagent device 31 stores various reagents. The data-processing unit 61 controls the reagent valve 32 to select one kind of reagent or more from the various reagents and then controls the reagent-injection component 33 to injected into the reaction chamber 11 through the inlet pipe 17. The data-processing unit 61 controls the reagent-ejection component 34 to eject the reacted reagent from the fluid cell 1 through the outlet pipe 18.

In the embodiment, the imaging unit 4 comprises: a light source 41, a compound lens 42 and an imaging component 43 (Not shown in FIG. 9). The light source 41 is fixed on one side of the fluid cell 1. The compound lens 42 and the imaging component 43 locate at the same side on the fluid cell 1. The lens components 42 and the imaging component 43 are used to image for the fluid cell 1. The light source 41 uses the mercury lamp light source. The compound lens 42 comprises: an incident light filter, a spectroscope, focus imaging lens assembly and emitted light filter. The imaging devices 43 uses a CCD probe.

In the embodiment, the signal intensity of sequencing reaction is strong. As a result, not only the ordinary mercury short-arc lamp can be used as the light source 41 to light for the fluid cell 1, but also the imaging component 43 can use the high specification CCD probe, such as the CCD probe from 4 million to 11 million pixels. The data-collecting unit 5 uses a reader (Sub-second time for full size), which can achieve high speed data collecting and improve the data throughput of the whole data. In the embodiment, the size and capacity of the fluid cell 1 can be selected flexibly to fit for the actual demands. The small size of fluid cell 1 makes the pixel of the whole imaging device 43 be used. To gain enough image data for gene sequencing, it is only needs 0.5-5 seconds to get a single image.

In the embodiment, the light source 41 and a compound lens 42 form the focus imaging system through a spectral-filter, irradiating on the DNA fragments 100 in the fluid cell 1. which also image multicolor image by auto-switching the optical filter model.

In the embodiment, the image data is analyzed and a database can also be created to manage the image data. Specifically, using the database to store and manage the image data gained from at least one site. Extract the data file about the signal strength after calculating the generating reaction.

In the embodiment, the data-processing unit 61 of the gene sequencing system controls the cycle sequencing of DNA fragments, which integrates the various functions about temperature controlling, reagent controlling, imaging, data collecting and processing.

Based on the above embodiments, the seventh embodiment is posed. According to the FIG. 2, the fluid cell 1 of gene sequencing system comprises: the drilled cover slide 14, the slide 15, the two of which is parallel opposite, and the gasket 16 located between the drilled cover slide 14 and the slide 15. The center of the gasket 16 has the through-hole 161. The two sides of the gasket 16 are tightly adhered to the drilled cover slide 14 and the slide 15. The through-hole 161, the inner side of the drilled cover slide 14 and the inner side of the slide 15 form a closed reaction chamber 11. As one inner side of reaction chamber 11, the inner side of the slide 15 fixes multiple DNA fragments 100 or beads 10.

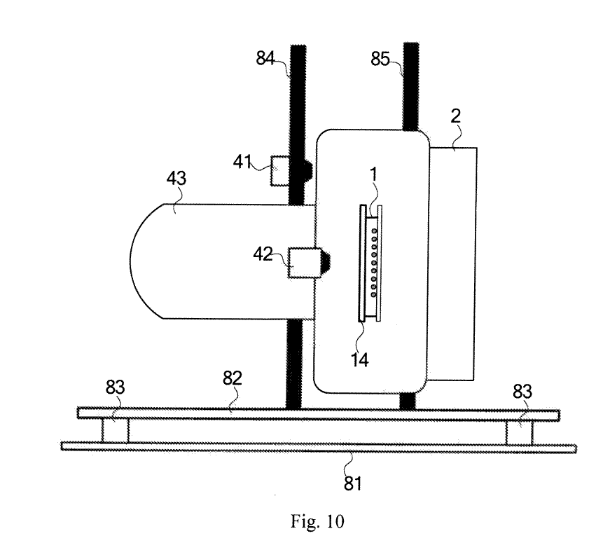

According to FIG. 10, the light source 41 is fixed on one side of the drilled cover slide 14. The imaging component 43 and the compound lens 42 are located at the same side of the fluid cell 1. Through the compound lens 42 and crossing the slide 15, the imaging component 43 images the sequencing reaction of reaction chamber 11.

The light source 41 also can be fixed on one side of the slide 15 on fluid cell 1. The imaging component 43 and the compound lens 42 are located on one side of the drilled cover slide 14 on the fluid cell 1. Through the compound lens 42 and the drilled cover slide 14, the imaging component 43 images the sequencing reaction of reaction chamber 11. Compared with the style through the slide 15 to image the sequencing reaction of reaction chamber 11, using this style makes the sequencing reaction more fully and the imaging result better, but the structure of sequencing reaction is more complex.

Based on the above embodiments, the eighth embodiment is posed. The FIG. 10 shows some parts of structure of gene sequencing system. The gene sequencing system further comprises a supporting unit 8 which fixes or arrays the various units, including the fluid cell 1 of the gene sequencing system, the temperature control unit 2, the reagent control unit 3 (Not shown in the figure), imaging unit 4 and so on, and make the units meet the position and working relationship. At the same time, it makes the gene sequencing system stable.

Specifically, the supporting unit 8 comprises: a backplane 81 of the system, a platform 82 set on the system backplane 81 and the at least two mainstays 83 to sure the platform 82 stability and reducing the shaking. The supporting unit 8 further comprises the first mainstay 84 and the second mainstay 85 which are plumb with the platform 82. the first mainstay 84 and the second mainstay 85, the two of which is parallel and exist side by side, are used for anchoring and supporting the fluid cell 1, the temperature control unit 2, the reagent control unit 3 (Not shown in the figure), the imaging unit 4 and so on. In order to operate the gene sequencing system automatically, it also can install a position adjusting equipment in the first mainstay 84 and the second mainstay 85 to adjust the relationship of position about the fluid cell 1, the temperature control unit 2, the reagent control unit 3 (Not shown in figure), the imaging unit 4 and so on. The position adjusting equipment can be made up by a knob with a screw.

In the embodiment, the process of the reaction is: the various different reagents of sequencing reaction are sub-packaged in the reagent device 31 by the reagent valve 32. When the reaction is going on, the control units 6 select the demanded reagents and control the reagent-injection component 33 to take quantitative reagent and inject the fluid cell 1. The temperature control unit 2 adjusts and controls the temperature of the fluid cell 1 to make sure the reaction go smoothly. After finished every reaction, the control units 6 select the washing reagents from the reagent device 31 to clean the fluid cell 1 and then change the reagents the next step need. After finished the whole sequencing reaction, the bases' information of DNA fragments is recorded by the imaging unit 4. Adjusting the position adjusting equipment to make the DNA fragments fixed at different position aim at the imaging unit 4 so as to collect the image data of the DNA fragments about each imaging area in the fluid cell 1 for storing and later analyzing.

In the embodiment and above embodiments, the fluid cell 1 can be located at level, upright or oblique. For example, in the second embodiments, the drilled cover slide 14 and the slide 15 which is parallel and opposite to the fluid cell 1 can be located at level, upright or certain angle, which makes the reagent inlet 12 and the reagent outlet 13 at the different level.

In order to avoid the bubble affecting imaging and ensure the imaging effect, the fluid cell 1 is set upright in the embodiments. The reagents inject into the reaction chamber 11 from the button and eject from the top.

The fluid cell 1 can also be mounted at the tilted position. The two ends are different in height. Reagents inject into reaction chamber 11 from the lower end and eject from the higher end, which makes the reagents full upon the reaction chamber 11 and DNA fragment 100 fully contact so as to ensure the DNA fragments 100 react thoroughly with the reagents and avoid air bubbles that may affect imaging. As a result, the quality of image data is better.

The above mentioned is only the optional embodiment of the invention, but doesn't limit the scope of patent about the present invention. Any using the content of specification and/or drawing of the invention to change the equal structure and process directly or indirectly, applying to the related technology fields, all above conditions are equal to contain in the scope of patent protection.

What is claimed is:

1. A fluid cell for sequencing reaction between DNA fragments and reagents, which fluid cell comprises:
a reaction chamber, one inner side of which is fixed with multiple DNA fragments;
a reagent inlet and a reagent outlet, which are separately located at each end of the other inner side of the reaction chamber and used for reagents flowing into and out from the reaction chamber.

2. The fluid cell according to claim 1, wherein, comprises: a drilled cover slide, a slide which is parallel and opposite to the drilled cover slide, and a gasket between the drilled cover slide and the slide;
the gasket has through-holes in its central section, and is pressed closely with the drilled cover slide and the slide on its two sides separately; the through-holes, inner side of the drilled cover slide and inner side of the slide form the closed reaction chamber;
as one side of the reaction chamber, the inner side of the slide is fixed with DNA fragments;
as the other side of the reaction chamber, two ends of the drilled cover slide have the first and second through-hole, which are used for connecting the reaction chamber with outside separately, and form the said reagent inlet and reagent outlet.

3. The fluid cell according to claim 1, wherein:
the through-hole of the gasket is narrow on both ends, and wide in middle, which looks like a leaf; said both ends of the through-hole separately correspond to the first through-hole and the second through-hole of the drilled cover slide, forming a reagent inlet and a reagent outlet.

4. The fluid cell according to claim 1, claim 2 or claim 3, wherein:
in the reaction chamber, the one side and/or the other side which fixed with DNA fragments are transparent.

5. The fluid cell according to claim 1, claim 2 or claim 3, wherein:
DNA fragments are attached to at least one bead which is fixed on the inner side of reaction chamber.

6. The fluid cell according to claim 1, claim 2 or claim 3, wherein:
DNA fragments are directly fixed on one inner side of the reaction chamber.

7. A gene sequencing reaction platform for controlling sequencing reaction between the DNA fragments and reagents, which platform comprises:
a fluid cell, the inner side of which is fixed with multiple DNA fragments;
a temperature control unit, used for heating and control the temperature of sequencing reaction;
a reagent control unit, used for controlling the reagents of sequencing reaction.

8. The gene sequencing reaction platform according to claim 7, wherein the temperature control unit comprises a heating component and a temperature-measuring component:
the temperature-measuring component, used for measuring temperature of sequencing reaction in real-time mode, and the measured temperature is used to control the heating component to heat the fluid cell;
the heating component, used for heating the fluid cell on the side which is fixed with DNA fragments, or on the other side.

9. The gene sequencing reaction platform according to claim 8, further comprising heating glasses which are used to heat the sequencing reaction;
the heating glasses, used for covering the outside of the fluid cell, which is heated by the heating component, and then heat and control temperature of the sequencing reaction in the reaction chamber through heat conduction of the fluid cell.

10. The gene sequencing reaction platform according to claim 7, wherein the reagent control unit comprises:
a reagent device, used for storing at least one kind of reagent;
a reagent valve, used for selecting one kind or several kinds of stored reagents from the reagent device;
a reagent-injection component, used for injecting selected reagents into the fluid cell;
a reagent-ejection component, used for ejecting reacted reagents from the fluid cell.

11. The gene sequencing reaction platform according to claim 7, claim 8, claim 9 or claim 10, wherein the said fluid cell is located at level, upright or oblique.

12. A gene sequencing system for controlling the sequencing reaction between DNA fragments and reagents, and collecting and processing the data of sequencing reaction, which system comprises:
a fluid cell, fixed with multiple DNA fragments and used for the gene sequencing reaction;
an imaging unit, used for imaging the signal on the said sequencing DNA fragments;
a data-collecting unit, used for collecting the image data;
control units, used for controlling the sequencing reaction and processing image data.

13. A gene sequencing system according to claim 12, wherein the fluid cell comprises:
a reaction chamber, in which multiple DNA fragments are fixed on one inner side;
a reagent inlet and a reagent outlet, which are separately located at each end of the other inner side of the reaction chamber and used for reagents flowing into and out from the reaction chamber.

14. A gene sequencing system according to claim 12, wherein:
the DNA fragments attached to at least one bead which is fixed on one inner side of the fluid cell; or
the DNA fragments are directly fixed on the inner side of the fluid cell.

15. A gene sequencing system according to claim 12, wherein the control units comprise:
a temperature control unit, used for heating and control temperature of the sequencing reaction;
a reagent control unit, used for controlling the reagents, comprising selecting, injecting and/or ejecting;
a data-processing unit, used for sending the control orders to the temperature unit and the reagent control unit and manage, analyze and process the image data.

16. A gene sequencing system according to claim 15, wherein the temperature control unit further comprises a heating component and a temperature-measuring component:
the temperature-measuring component, used for measuring temperature of sequencing reaction in real-time mode and sends the measured temperature to the data-processing unit to analyze the measured temperature and generate temperature controlling orders;
the heating component, used for heating the fluid cell according to the temperature controlling orders from the data-processing unit.

17. A gene sequencing system according to claim 13 or claim 14, wherein the imaging unit comprises:
a light source, which is fixed on one side of the fluid cell;
an imaging component and a compound lens, which are located on the same side of the fluid cell;
the imaging component, used for taking pictures of the fluid cell through the compound lens.

18. A gene sequencing system according to claim 13 or claim 14, wherein the fluid cell is located at level, upright or oblique.

19. A gene sequencing system according to claim 13 or claim 14, wherein the gene sequencing system further comprises:
a supporting unit, used for anchoring and/or arranging the fluid cell and the imaging unit separately, in order to make the gene sequencing system stable.

20. A gene sequencing system according to claim 19, wherein the supporting unit further comprises:
a position adjusting device, used for changing the distance between the fluid cell and the imaging component.
